(19) United States
(12) Patent Application Publication
(10) Pub. No.: US 2016/0101090 A1
(43) Pub. Date: Apr. 14, 2016

(54) INHIBITION OF TRK KINASE MEDIATED TUMOR GROWTH AND DISEASE PROGRESSION

(71) Applicant: DECIPHERA PHARMACEUTICALS, LLC, Lawrence, KS (US)

(72) Inventors: Daniel L. FLYNN, Lawrence, KS (US); Michael D. KAUFMAN, Lawrence, KS (US); Bryan D. SMITH, Lawrence, KS (US)

(21) Appl. No.: 14/883,072

(22) Filed: Oct. 14, 2015

Related U.S. Application Data
(60) Provisional application No. 62/063,660, filed on Oct. 14, 2014.

(51) Int. Cl. A61K 31/44; A61K 45/06

(52) U.S. Cl. CPC A61K 31/44; A61K 45/06

(57) ABSTRACT

It has been shown that Compound 1 unexpectedly and potently inhibits TRK kinases, including all three forms of TRK: NTRK1, NTRK2, and NTRK3. Additionally it has been shown that Compound 1 potently inhibits oncogenic mutated forms of TRK kinases, including fusion proteins. By way of exemplification, Compound 1 potently inhibits the NTRK1 oncogenic fusion protein TPM3/NTRK1 in cellular assays. Compound 1 inhibits TRK kinase mediated tumor growth in vivo in a TPM3/NTRK1 xenograft model.

### CROSS REFERENCE TO RELATED APPLICATIONS

This application claims priority to U.S. Provisional Application No. 62/063,660, filed Oct. 14, 2014, the contents of which are incorporated herein by reference in their entireties.

### DESCRIPTION OF THE TEXT FILE SUBMITTED ELECTRONICALLY

The content of the text file submitted electronically herewith is incorporated herein by reference in its entirety: A computer readable format copy of the Sequence Listing (filename: DECP_067_01US_SeqList_ST25.txt; date recorded Oct. 13, 2015: file size 10 KB).

### BACKGROUND

Kinase fusion proteins are known to be causative of a variety of cancers (Vaishnavi 2015; Stransky 2014). The most frequently reported kinase fusions that are driver mutations in cancer are receptor tyrosine kinase fusions, wherein chromosomal translocation gives rise to a constitutively active mutant kinase wherein the catalytic kinase domain (C-terminal region of a receptor tyrosine kinase) is fused with an N-terminal region derived from another gene. Typically the N-terminal region promotes constitutive activation of the kinase domain usually by promoting fusion protein dimerization (Stranksy 2014).

Fusion proteins have been reported that are the result of chromosomal translocation of the kinase domain of NTRK1, NTRK2, or NTRK3 with a variety of N-terminal fusion partners. NTRK1 gene fusions have been demonstrated to be driver mutations in lung adenocarcinoma, cholangiocarcinoma, colorectal cancer, papillary thyroid cancer, spitzoid neoplasms, and glioblastoma. NTRK2 gene fusions have been demonstrated to be driver mutations in sarcomas, astrocytomas, lung adenocarcinoma, and head and neck cancer. NTRK3 gene fusions have been demonstrated to be driver mutations in lower grade glioma, secretory breast cancer, papillary thyroid cancer, acute myeloid leukemia, congenital mesoblastic nephroma, congenital fibrosarcoma, acute lymphoblastic leukemia, colon adenocarcinoma, thyroid carcinoma, cutaneous melanoma, head and neck cancer, and pediatric glioma (Vaishnavi 2015).

Specific TRK fusion proteins that have been reported include MPRIP-NTRK1, CD74-NTRK1, RFWD2-NTRK1, and SQSTM1-NTRK1 fusions in lung adenocarcinoma (Vaishnavi 2013, Cuesta 2014, Patel 2015); TPM3-NTRK1, TFG-NTRK1, and TPR-NTRK1 in colorectal and thyroid cancers (Alberti 2003; Greco 2010, Martin-Zanca 1986); TPM3-NTRK1 in sarcoma (Stranksy 2014); RABGAP1L-NTRK1 in cholangiocarcinoma (Ross 2014); LMNA-NTRK1 and TP53-NTRK1 in spitzoid tumors (Wiesner 2014); NFASC-NTRK1 in glioblastoma (Kim 2014); PAN3-NTRK2 fusions in head and neck cancer (Stransky 2014; AFAP1-NTRK2 fusion in low grade glioma (Stransky 2014); TRIM24-NTRK2 in lung adenocarcinoma (Stransky 2014); and ETV6-NTRK3 in congenital fibrosarcoma and secretory breast cancer (Knezevich 1998; Ricarte-Filho 2013, Tognon 2002).

In addition to TRK kinase fusion proteins, other forms of TRK kinase have been demonstrated to cause cancers. A deletion mutation in NTRK1 has been shown to be causative of acute myeloid leukemia (Reuther 2000). Inactivation of NTRK1 has been shown to sensitize pancreatic tumors to gemcitabine (Liu 2007). Enhanced expression of NGF/NTRK1 was shown to play a role in perineural invasion and the pain syndrome in human pancreatic cancer (Zhu 1999). Elevated TRK kinase signaling has also been demonstrated in neuroblastoma (Brodeur 2009).

There is a need in the art for improved treatment of cancers associated with one or more TRK kinase mutation, TRK kinase overexpression, and/or one or more TRK kinase fusion protein.

### SUMMARY OF THE INVENTION

In one aspect, the present disclosure provides compositions and methods for treating cancers associated with overexpression of a TRK kinase, one or more mutations of a TRK kinase, and/or one or more TRK kinase fusion proteins. In another aspect, Compound 1 or a pharmaceutically acceptable salt thereof is administered to a subject having or suspected of having cancer, wherein tumor growth, survival, or progression of the cancer is caused by an overexpression of a TRK kinase, mutation of a TRK kinase, or a TRK kinase fusion protein.

In one embodiment, Compound 1 or a pharmaceutically acceptable salt thereof is administered to a subject having or suspected of having cancer, wherein tumor growth, survival, or progression is caused by a NTRK1 fusion protein. In a further embodiment, the NTRK1 fusion protein is selected, without limitation, from MPRIP-NTRK1, CD74-NTRK1, RFWD2-NTRK1, SQSTM1-NTRK1, TPM3-NTRK1, TFG-NTRK1, TPR-NTRK1, RABGAP1L-NTRK1 LMNA-NTRK1, TP53-NTRK1, NFASC-NTRK1, and PEAR1-NTKR1.

In another embodiment, Compound 1 or a pharmaceutically acceptable salt thereof is administered to a subject having or suspected of having cancer, wherein tumor growth, survival, or progression is caused by a NTRK2 fusion protein. In a further embodiment, the NTRK2 fusion protein is selected, without limitation, from PAN3-NTRK2, AFAP1-NTRK2, TRIM24-NTRK2, QK1-NTRK2, NACC2-NTRK2, VCL-NTRK2, and AGBL4-NTRK2.

In another embodiment, Compound 1 or a pharmaceutically acceptable salt thereof is administered to a subject having or suspected of having cancer, wherein tumor growth, survival, or progression is caused by a NTRK3 fusion protein. In a further embodiment, the NTRK3 fusion protein is selected, without limitation, from ETV6-NTRK3 and BTBD1-NTRK3.

In another embodiment, Compound 1 or a pharmaceutically acceptable salt thereof is administered to a subject having or suspected of having cancer, wherein tumor growth, survival, or progression is caused by a mutation in a TRK kinase. In a further embodiment, the mutation may include an NTRK1 deletion mutation in acute myeloid leukemia.

In another embodiment, Compound 1 or a pharmaceutically acceptable salt thereof is administered to a subject having or suspected of having cancer, wherein tumor growth, survival, or progression is caused by overexpression of a wild-type TRK kinase. In a further embodiment, the TRK kinase is overexpressed in pancreatic cancer or neuroblastoma.

In another embodiment, Compound 1 or a pharmaceutically acceptable salt thereof is administered to a subject having or suspected of having cancer, wherein tumor growth, survival, or progression is caused by a TRK fusion protein and wherein the cancer is lung adenocarcinoma, cholangiocarcinoma, colorectal cancer, colon adenocarcinoma, papillary thyroid cancer, spitzoid neoplasms, glioblastoma, sarcomas, congenital fibrosarcoma, astrocytomas, head and neck cancer, low grade glioma, secretory breast cancer, acute myeloid leukemia, congenital mesoblastic nephroma, acute lymphoblastic leukemia, thyroid carcinoma, cutaneous melanoma, pediatric glioma, neuroblastoma, or pancreatic cancer.

In another embodiment, Compound 1 or a pharmaceutically acceptable salt thereof is administered to a subject having or suspected of having cancer, as a single agent or in combination with other cancer targeted therapeutic agents, cancer-targeted biologicals, or chemotherapeutic agents.

In some embodiments, the effective amount of N-(4-(2-(cyclopropanecarboxamido)pyridin-4-yloxy)-2,5-difluorophenyl)-N′-(4)cyclopropane-1,1-dicarboxamide, or pharmaceutically acceptable salt thereof is administered to the subject orally.

### BRIEF DESCRIPTION OF THE DRAWINGS

FIG. 1, panel A is a line graph showing that treatment with Compound 1 results in a delay in tumor growth in a KM-12 TPM3-NTRK1 xenograft model. 15 mg/kg, 7.5 mg/kg, and 3.75 mg/kg doses of Compound 1 (PO, BID) were tested relative to vehicle control. The delay in tumor growth was statistically significant at doses of 15 mg/kg and 7.5 mg/kg.

FIG. 1, panel B is a bar graph showing the percent inhibition of NTRK1 phosphorylative activation after a single oral dose of Compound 1 at 15 mg/kg or 7.5 mg/kg.

FIG. 2, panel A is a line graphs showing that treatment with 15 mg/kg (PO, BID) Compound 1 results in a delay of tumor growth in a NIH-3T3 ETV6-NTRK3 xenograft model.

FIG. 2, panel B is a bar graph showing the percent inhibition of ETV6-NTRK3 phosphorylative activation after a single oral dose of Compound 1 at 15 mg/kg.

### DETAILED DESCRIPTION

It has been found that N-(4-(2-(cyclopropanecarboxamido)pyridin-4-yloxy)-2,5-difluorophenyl)-N′-(4)cyclopropane-1,1-dicarboxamide unexpectedly inhibits wild-type and oncogenic fusion protein forms of NTRK1, NTRK2, and NTRK3 kinases. The present disclosure provides compositions and methods for treating cancer by inhibiting TRK kinase mediated tumor growth and disease progression comprising administering to a subject in need thereof an effective amount of N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof.

Thus, in one aspect, the present disclosure provides methods and compositions for treating cancers associated with overexpression of a TRK kinase, one or more mutations of a TRK kinase, and/or one or more TRK kinase fusion proteins. The compositions and methods provided herein inhibit or prevent tumor growth, survival, and/or progression.

Compound 1 as used herein refers to the compound N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof, whose structure is below:

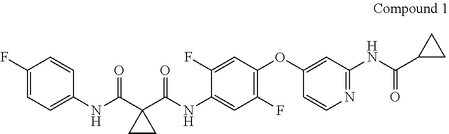 [SMILES: O=C(Nc1cc(Oc2cc(F)c(NC(=O)C3(C(=O)Nc4ccc(F)cc4)CC3)cc2F)ccn1)C1CC1]

Methods of making Compound 1 are disclosed in U.S. Pat. No. 8,637,672 the contents of which are incorporated herein by reference. The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

In some embodiments, the cancer is selected from lung adenocarcinoma, cholangiocarcinoma, colorectal cancer, colon adenocarcinoma, papillary thyroid cancer, spitzoid neoplasms, glioblastoma, sarcomas, congenital fibrosarcoma, astrocytomas, head and neck cancer, low grade glioma, secretory breast cancer, acute myeloid leukemia, congenital mesoblastic nephroma, acute lymphoblastic leukemia, thyroid carcinoma, cutaneous melanoma, pediatric glioma, neuroblastoma, pancreatic cancer, gastrointestinal stromal tumor, ovarian cancer, renal cancer, hepatic cancer, cervical carcinoma, non small cell lung cancer, mesothelioma, colon cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, breast cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, retinoblastoma, and neuroendocrine tumor.

In some embodiments, the fusion proteins of the present disclosure are the result of chromosomal translocation of the kinase domain of NTRK1, NTRK2, or NTRK3 with a variety of N-terminal fusion partners. TRK fusion proteins include, but are not limited to, MPRIP-NTRK1, CD74-NTRK1, RFWD2-NTRK1, SQSTM1-NTRK1, TPM3-NTRK1, TFG-NTRK1, TPR-NTRK1, TPM3-NTRK1, RABGAP1L-NTRK1, LMNA-NTRK1, TP53-NTRK1, NFASC-NTRK1, PAN3-NTRK2, AFAP1-NTRK2, TRIM24-NTRK2, and ETV6-NTRK3. In addition to fusion proteins, other forms of TRK kinase have been demonstrated to cause cancers. For example, overexpression of a TRK kinase and/or one or more mutations of a TRK kinase have been demonstrated to cause cancers. Mutations may include substitutional, insertional, and/or deletional variants of TRK.

The terms “patient” and “subject” are used interchangeably herein. In one embodiment, the subject may be a mammal, such as a rodent, a feline, a canine, and a primate. Preferably, a subject is a human. In one embodiment, the compounds and additional therapeutics provided herein may be administered by any suitable route, independently selected from oral, parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intratympanic, intrauterine, intravesical, intravitreal, bolus, subconjunctival, vaginal, rectal, buccal, sublingual, intranasal, intratumoral, and transdermal. In further embodiments, the effective amount of N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof, is administered to the subject orally.

The term “pharmaceutically acceptable salt” embraces salts commonly used to form salts of free bases. The nature of the salt is not critical, provided that it is pharmaceutically-acceptable. The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Suitable pharmaceutically acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, and heterocyclyl containing carboxylic acids and sulfonic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” with regard to a subject, refers to improving at least one symptom of the subject's disorder. Treating can be preventing, curing, improving, or at least partially ameliorating the disorder.

The terms “effective amount” and “therapeutically effective amount” are used interchangeably in this disclosure and refer to an amount of a compound that, when administered to a subject, is capable of reducing a symptom of a disorder in a subject. The actual amount which comprises the “effective amount” or “therapeutically effective amount” will vary depending on a number of conditions including, but not limited to, the particular disorder being treated, the severity of the disorder, the size and health of the patient, and the route of administration. A skilled medical practitioner can readily determine the appropriate amount using methods known in the medical arts.

In some embodiments, N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof, is administered to the subject as a single agent. In other embodiments, N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof, is administered to the subject in combination with an additional therapeutic agent. Additional therapeutic agents include other cancer targeted therapeutic agents, cancer-targeted biologicals, immunotherapeutics, and/or chemotherapeutic agents.

In some embodiments, the additional chemotherapeutic agent is an anti-tubulin agent. In further embodiments, the anti-tubulin agent is selected from paclitaxel, docetaxel, abraxane, and eribulin. In some embodiments, the immunotherapeutic agent is an anti-CTLA-4 agent, an anti-PD agent, an anti-PDL agent, or an IDO inhibitor. In some embodiments, the immunotherapy agent is selected from ipilimumab, pembrolizumab, nivolumab, atezolizumab, avelumab, MEDI4736, indoximod, INCB024360, and epacadostat. In some embodiments, cancer-targeted biologicals may include monoclonal antibodies, kinase inhibitors and inhibitors of growth factors and their receptors, gene therapy agents, cell therapy, e.g., stem cells, or any combination thereof.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs. The initial definition provided for a group or term provided in this disclosure applies to that group or term throughout the present disclosure individually or as part of another group, unless otherwise indicated.

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

### Example 1

### Evaluation of Compound 1 as an Inhibitor NTRK1 (SEQ ID No. 1), NTRK2 (SEQ ID No. 2), and NTRK3 (SEQ ID No. 3) Recombinant Kinases

Activity of NTRK1, NTRK2, and NTRK3 kinases was determined by following the production of ADP from the kinase reaction through coupling with the pyruvate kinase/lactate dehydrogenase system [Schindler 2000]. In this assay, the oxidation of NADH (thus the decrease at A_{340 nm}) was continuously monitored spectrophotometrically. The reaction mixtures (100 μL) contained kinase [NTRK1 (Invitrogen) (4.7 nM), NTRK2 (Invitrogen) (4 nM), or NTRK3 (Invitrogen) (1.8 nM)], polyEY (1 mg/mL), MgCl₂ (18 mM), DTT (0.5 mM), pyruvate kinase (4 units), lactate dehydrogenase (7 units), phosphoenol pyruvate (1 mM), and NADH (0.28 mM) and ATP (1 mM for NTRK1 and NTRK2; 0.25 mM for NTRK3) in 90 mM Tris buffer containing 0.2% octyl-glucoside and 1% DMSO, pH 7.5. The inhibition reaction was started by mixing serial diluted test Compound 1 with the above reaction mixture. The absorption at 340 nm was monitored continuously for 4 hours at 30° C. on a plate reader (BioTek). The reaction rate was calculated using the 2 to 4 h time frame. Percent inhibition was obtained by comparison of reaction rate with that of a control (i.e. with no test compound). IC₅₀ value for Compound 1 was calculated from a series of percent inhibition values determined at a range of inhibitor concentrations using software routines as implemented in the GraphPad Prism software package.

Compound 1 inhibited recombinant NTRK1 kinase activity with an IC₅₀ value of 0.71 nM, inhibited NTRK2 kinase with an IC₅₀ of 4.6 nM, and inhibited NTRK3 kinase with an IC₅₀ of 0.83 nM (Table 1).

TABLE 1
Compound 1 potently inhibits TRK kinases biochemically and blocks
proliferation in oncogenic TRK cellular assays (IC50 values, nM)

| TRK Kinase | TRK Cellular |
|---|---|
| NTRK1 | TPM3-NTRK1 |
| 0.71 ± 0.33 nM | KM-12/CRC |
| | 3.8 ± 1.8 nM |
| NTRK2 | |
| 4.6 ± 0.4 nM | |
| NTRK3 | ETV6-NTRK3 |
| 0.83 ± 0.39 nM | Transfected NIH-3T3 |
| | 0.44 ± 0.26 nM |

NTRK1 protein sequence used for screening (SEQ ID No. 1)

MKCGRRNKFGINRPAVLAPEDGLAMSLHFMTLGGSSLSPTEGKGSGLQGH
IIENPQYFSDACVHHIKRRDIVLKWELGEGAFGKVFLAECHNLLPEQDKM
LVAVKALKEASESARQDFQREAELLTMLQHQHIVRFFGVCTEGRPLLMVF
EYMRHGDLNRFLRSHGPDAKLLAGGEDVAPGPLGLGQLLAVASQVAAGMV
YLAGLHFVHRDLATRNCLVGQGLVVKIGDFGMSRDIYSTDYYRVGGRTML
PIRWMPPESILYRKFTTESDVWSFGVVLWEIFTYGKQPWYQLSNTEAIDC
ITQGRELERPRACPPEVYAIMRGCWQREPQQRHSIKDVHARLQALAQAPP
VYLDVLGKGVEACQLGTDDYDIPTTHHHHHH

NTRK2 protein sequence used for screening (SEQ ID No. 2)

MVIENPQYEGITNSQLKPDTFVQHIKRHNIVLKRELGEGAFGKVFLAECY
NLCPEQDKILVAVKTLKDASDNARKDEHREAELLTNLQHEHIVKFYGVCV
EGDPLIMVFEYMKHGDLNKFLRAHGPDAVLMAEGNPPTELTQSQMLHIAQ
QIAAGMVYLASQHFVHRDLATRNCLVGENLLVKIGDEGMSRDVYSTDYYR
VGGHTMLPIRWMPPESIMYRKFTTESDVWSLGVVLWEIFTYGKQPWYQLS
NNEVIECITQGRVLQRPRTCPQEVYELMLGCWQREPHMRKNIKGIHTLLQ
NLAKASPVYLDILGKGGRADPAFLYKVVRMNEDLGKPIPNPLLGLDSTRT
GHHHHHH

NTRK3 protein sequence used for screening (SEQ ID No. 3)

MVIENPQYFRQGHNCHKPDTYVQHIKRRDIVLKRELGEGAFGKVFLAECY
NLSPTKDKMLVAVKALKDPTLAARKDFQREAELLTNLQHEHIVKFYGVCG
DGDPLIMVFEYMKHGDLNKFLRAHGPDAMILVDGQPRQAKGELGLSQMLH
IASQIASGMVYLASQHFVHRDLATRNCLVGANLLVKIGDFGMSRDVYSTD
YYRVGGHTMLPIRWMPPESIMYRKFTTESDVWSFGVILWEIFTYGKQPWF
QLSNTEVIECITQGRVLERPRVCPKEVYDVMLGCWQREPQQRLNIKEIYK
ILHALGKATPIYLDILGKGGRADPAFLYKVVRMNEDLGKPIPNPLLGLDS
TRTGHHHHHH

### Example 2

### Evaluation of Compound 1 as an Inhibitor of Cellular Proliferation in the KM-12 Colorectal Cancer Cell Line Harboring the TPM3-NTRK1 Fusion Protein

### KM-12 Cell Culture

KM-12 cells were obtained from the Division of Cancer Treatment and Diagnosis Tumor Repository, National Cancer Institute (Frederick, Md.). Briefly, cells were grown in RPMI 1640 media supplemented with 10% characterized fetal bovine serum and 1% Penicillin/Streptomycin/L-glutamine solution (Invitrogen, Carlsbad, Calif.) at 37 degrees Celsius, 5% CO₂, and 95% humidity. Cells were allowed to expand until reaching 70-95% confluency at which point they were subcultured or harvested for assay use.

### KM-12 Cell Proliferation Assay

A serial dilution of test compound was dispensed into a 384-well black clear bottom plate (Corning, Corning, N.Y.). One thousand two hundred fifty cells were added per well in 50 μL complete growth medium. Plates were incubated for 67 hours at 37 degrees Celsius, 5% CO₂, and 95% humidity. At the end of the incubation period 10 μL of a 440 μM solution of resazurin (Sigma, St. Louis, Mo.) in PBS was added to each well and incubated for an additional 5 hours at 37 degrees Celsius, 5% CO₂, and 95% humidity. Plates were read on a Synergy2 reader (Biotek, Winooski, Vt.) using an excitation of 540 nM and an emission of 600 nM. Data was analyzed using Prism software (Graphpad, San Diego, Calif.) to calculate IC₅₀ values.

Compound 1 exhibited an IC₅₀ value of 3.8 nM for inhibiting proliferation of KM-12 colorectal cancer cells expressing the TPM3/NTRK1 oncogenic fusion protein (Table 1).

### Example 3

### Evaluation of Compound 1 as an Inhibitor of Transfected NIH3T3 Cell Proliferation Driven by ETV6-NTRK3 Fusion Kinase

### Transfected ETV6-NTRK3 Cell Culture

Transfected NIH-3T3 ETV6-NTRK3 cells were obtained from the laboratory of James Fagin, MD (Memorial Sloan Kettering Cancer Center). Briefly, cells were grown in DMEM media supplemented with 10% characterized fetal bovine serum, 1% Penicillin/Streptomycin/L-glutamine solution (Life Technologies, Carlsbad, Calif.) and 1 μg/mL puromycin (Invitrogen, Carlsbad, Calif.) at 37 degrees Celsius, 5% CO₂, and 95% humidity. Cells were allowed to expand until reaching approximately 75% confluency at which point they were subcultured or harvested for assay use.

### Transfected NIH-3T3 ETV6-NTRK3 Cell Proliferation Assay

A serial dilution of test compound was dispensed into a 96-well black clear bottom plate (Corning, Corning, N.Y.). Fifteen thousand cells were added per well in 200 μL medium (DMEM media supplemented with 0.5% characterized fetal bovine serum (Life Technologies, Carlsbad, Calif.). Plates were incubated for six days at 37 degrees Celsius, 5% CO₂, and 95% humidity. At the end of the incubation period 40 μL of a 440 μM solution of resazurin (Sigma, St. Louis, Mo.) in PBS was added to each well and incubated for an additional 5 hours at 37 degrees Celsius, 5% CO₂, and 95% humidity. Plates were read on a Synergy2 reader (Biotek, Winooski, Vt.) using an excitation of 540 nM and an emission of 600 nM. Data was analyzed using Prism software (Graphpad, San Diego, Calif.) to calculate IC₅₀ values.

Compound 1 exhibited an IC₅₀ of 0.44 nM for inhibition of transfected NIH-3T3 ETV6-NTRK3 cell proliferation driven by the ETV6-NTRK3 kinase fusion protein (Table 1)

### Example 4

### Evaluation of Compound 1 as an Inhibitor of Cellular Phosphorylative Activation of NTRK1 Kinase in K562 Chronic Myeloid Leukemia Cells

### K562 Cell Culture

K562 cells (catalog #CCL-243) were obtained from the American Type Culture Collection (ATCC; Manassas, Va.). Briefly, K562 cells were grown in suspension in IMDM medium supplemented with 10% characterized fetal bovine serum and 1% Penicillin-Streptomycin-L-glutamine solution (Invitrogen, Carlsbad, Calif.) at 37 degrees Celsius, 5% CO₂, and 95% humidity. Cells were allowed to expand until reaching one to three million cells per milliliter at which point they were subcultured or harvested for assay use.

### K562 Phospho-NTRK1 Western Blot

One million cells in serum-free IMDM media were added per well in a 24-well tissue-culture treated plate. A serial dilution of test compound was added to cells and plates were incubated for 4 hours at 37 degrees Celsius, 5% CO2, and 95% humidity. Cells were then stimulated for 10 minutes with 100 ng/mL NGF (R&D Systems, Minneapolis, Minn.). Cells pelleted by centrifugation, washed with PBS, then lysed. Cell lysates were separated by SDS-PAGE and transferred to PVDF. Phospho-NTRK1 (Tyr674/675) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total NTRK1 using an antibody from Santa Cruz Biotech (Santa Cruz, Calif.). IC₅₀ values were calculated using Prism software (Graphpad, San Diego, Calif.).

Compound 1 inhibited NTRK1 phosphorylation in K562 cells with an IC₅₀ value of 0.69 nM (Table 2).

TABLE 2
Inibition of phosphorylation of cellular
TRK kinases by Compound 1

| Kinase | Cell Line | IC₅₀, nM | n |
|---|---|---|---|
| NTRK1 | K562 | 0.69 | 2 |
| NTRK1 | SK-N-SH | 1.0 ± 0.5 | 4 |
| TPM3-NTRK1 | KM-12 | 1.4 | 2 |
| ETV6-NTRK3 | NIH 3T3 | 0.47 ± 0.42 | 3 |
| NTRK2 | SK-N-SH* | 0.24 ± 0.14 | 3 |

*SK-N-SH cells were differentiated with all-trans retinoic acid prior to assay to induce NTRK2 expression

### Example 5

### Evaluation of Compound 1 as an Inhibitor of Cellular Phosphorylative Activation of NTRK1 Kinase in SK-N-SH Neuroblastoma Cells

### SK-N-SH Cell Culture

SK-N-SH cells (catalog #HTB-11) were obtained from the American Type Culture Collection (ATCC; Manassas, Va.). Briefly, SK-N-SH cells were grown in MEM medium supplemented with 10% characterized fetal bovine serum and 1% Penicillin-Streptomycin-L-glutamine solution (Invitrogen, Carlsbad, Calif.) at 37 degrees Celsius, 5% CO₂, and 95% humidity. Cells were allowed to expand until reaching 70-95% confluency at which point they were subcultured or harvested for assay use.

### SK-N-SH Phospho-NTRK1 Western Blot

Two hundred fifty thousand cells in growth medium were added per well in a 24-well tissue-culture treated plate. The plate was incubated overnight at 37 degrees Celsius, 5% CO₂, and 95% humidity. The next day, growth media was aspirated, cells were washed with serum-free MEM media, and one milliliter serum-free MEM media was added per well. A serial dilution of test compound was added to the cells, and plates were incubated for 4 hours at 37 degrees Celsius, 5% CO₂, and 95% humidity. Cells were then stimulated for 10 minutes with 100 ng/mL NGF (R&D Systems, Minneapolis, Minn.). Media was then aspirated, cells were washed with PBS, and then lysed. Cell lysates were separated by SDS-PAGE and transferred to PVDF. Phospho-NTRK1 (Tyr674/675) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total NTRK1 using an antibody from Santa Cruz Biotech (Santa Cruz, Calif.). IC₅₀ values were calculated using Prism software (Graphpad, San Diego, Calif.).

Compound 1 inhibited NTRK1 phosphorylation in SK-N-SH cells with an IC₅₀ value of 1.0 nM (Table 2).

### Example 6

### Evaluation of Compound 1 as an Inhibitor of Cellular Phosphorylative Activation of TPM3-NTRK1 Fusion Kinase in KM-12 Cells

### KM-12 Cell Culture

KM-12 cells were obtained from the Division of Cancer Treatment and Diagnosis Tumor Repository, National Cancer Institute (Frederick, Md.). Briefly, cells were grown in RPMI 1640 media supplemented with 10% characterized fetal bovine serum and 1% Penicillin/Streptomycin/L-glutamine solution (Invitrogen, Carlsbad, Calif.) at 37 degrees Celsius, 5% CO₂, and 95% humidity. Cells were allowed to expand until reaching 70-95% confluency at which point they were subcultured or harvested for assay use.

### KM-12 Phospho-NTRK1 Western Blot

Two hundred fifty thousand cells in growth medium were added per well in a 24-well tissue-culture treated plate. The plate was incubated overnight at 37 degrees Celsius, 5% CO₂, and 95% humidity. The next day, a serial dilution of test compound was added to the cells, and plates were incubated for 4 hours at 37 degrees Celsius, 5% CO₂, and 95% humidity. Media was then aspirated, cells were washed with PBS, and then lysed. Cell lysates were separated by SDS-PAGE and transferred to PVDF. Phospho-NTRK1 (Tyr674/675) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total NTRK1 using an antibody from Santa Cruz Biotech (Santa Cruz, Calif.). IC₅₀ values were calculated using Prism software (Graphpad, San Diego, Calif.).

Compound 1 inhibited TPM3-NTRK1 phosphorylation in KM-12 cells with an IC₅₀ value of 1.4 nM (Table 2).

### Example 7

### Evaluation of Compound 1 as an Inhibitor of Cellular Phosphorylative Activation of ETV6-NTRK3 Fusion Kinase in Transfected NIH-3T3 Cells

### Transfected NIH-3T3 ETV6-NTRK3 Cell Culture

Transfected NIH-3T3 ETV6-NTRK3 cells were obtained from the laboratory of James Fagin, MD (Memorial Sloan Kettering Cancer Center). Briefly, cells were grown in DMEM media supplemented with 10% characterized fetal bovine serum, 1% Penicillin/Streptomycin/L-glutamine solution (Life Technologies, Carlsbad, Calif.) and 1 μg/mL puromycin (Invitrogen, Carlsbad, Calif.) at 37 degrees Celsius, 5% CO₂, and 95% humidity. Cells were allowed to expand until reaching approximately 75% confluency at which point they were subcultured or harvested for assay use.

### Transfected NIH-3T3 ETV6-NTRK3 Phospho-NTRK3 Western Blot

Eighty thousand cells in media (DMEM media supplemented with 0.5% FBS and 1% Penicillin/Streptomycin/L-glutamine solution) were added per well in a 12-well tissue-culture treated plate. The plate was incubated for three days at 37 degrees Celsius, 5% CO₂, and 95% humidity. Next, media was aspirated, and two milliliters serum-free DMEM was added. A serial dilution of test compound was added to the cells, and plates were incubated for 4 hours at 37 degrees Celsius, 5% CO₂, and 95% humidity. Media was then aspirated, cells were washed with PBS, and then lysed. Cell lysates were separated by SDS-PAGE and transferred to PVDF. Phospho-NTRK3 (Tyr516) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. IC₅₀ values were calculated using Prism software (Graphpad, San Diego, Calif.).

Compound 1 inhibited ETV6-NTRK3 phosphorylation in transfected NIH-3T3 cells with an IC₅₀ value of 0.47 nM (Table 2).

### Example 8

### Evaluation of Compound 1 as an Inhibitor of Cellular Phosphorylative Activation of NTRK2 in SK-N-SH Cells

### SK-N-SH Cell Culture

SK-N-SH cells (catalog #HTB-11) were obtained from the American Type Culture Collection (ATCC; Manassas, Va.). Briefly, SK-N-SH cells were grown in MEM medium supplemented with 10% characterized fetal bovine serum and 1% Penicillin-Streptomycin-L-glutamine solution (Invitrogen, Carlsbad, Calif.) at 37 degrees Celsius, 5% CO₂, and 95% humidity. Cells were allowed to expand until reaching 70-95% confluency at which point they were subcultured. To induce expression of NTRK2, cells were grown in growth medium containing 10 μM all-trans retinoic acid for ten to fourteen days prior to harvesting cells for assay use.

SK-N-SH phospho-NTRK1 Western Blot

Two hundred fifty thousand cells differentiated with 10 μM all-trans retinoic acid in growth medium were added per well in a 12-well tissue-culture treated plate. The plate was incubated overnight at 37 degrees Celsius, 5% CO₂, and 95% humidity. The next day, growth media was aspirated, cells were washed with serum-free MEM media, and one milliliter serum-free MEM media was added per well. A serial dilution of test compound was added to the cells, and plates were incubated for 4 hours at 37 degrees Celsius, 5% CO₂, and 95% humidity. Cells were then stimulated for five minutes with 100 ng/mL BDNF (R&D Systems, Minneapolis, Minn.). Media was then aspirated, cells were washed with PBS, and then lysed. Cell lysates were separated by SDS-PAGE and transferred to PVDF. Phospho-NTRK2 (Tyr706/707) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total NTRK2 using an antibody from Santa Cruz Biotech (Santa Cruz, Calif.). IC₅₀ values were calculated using Prism software (Graphpad, San Diego, Calif.).

Compound 1 inhibited NTRK2 phosphorylation in SK-N-SH cells with an IC₅₀ value of 0.24 nM (Table 2).

### Example 9

### Evaluation of Compound 1 in the TPM3-NTRK1 Transformed KM-12 Xenograft Model

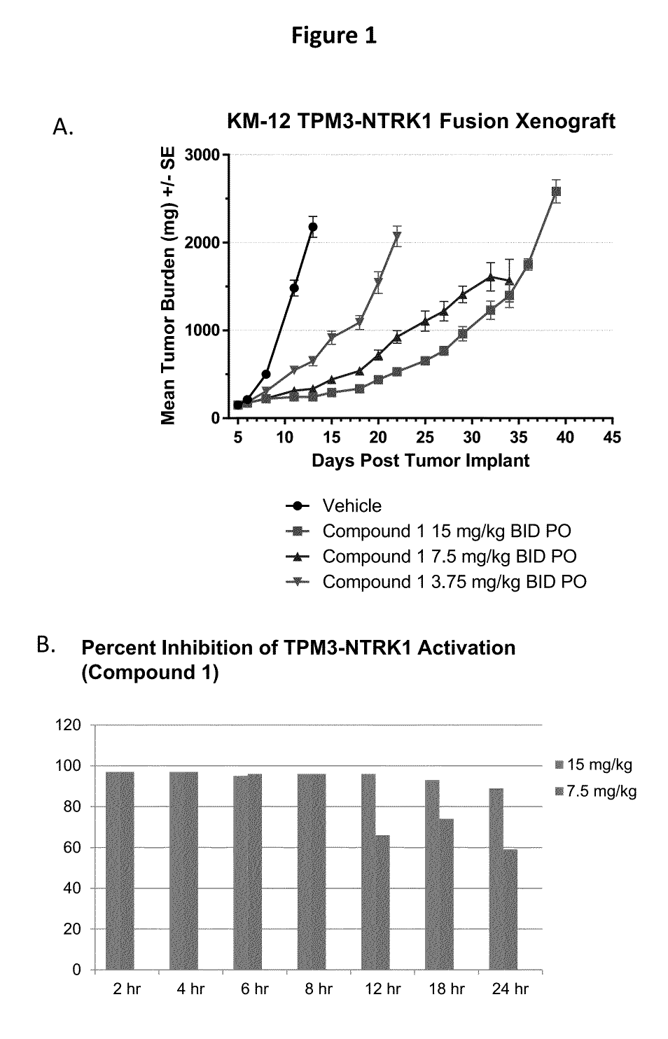

Compound 1 was evaluated for single-agent efficacy in the KM-12 TPM3-NTRK1 xenograft model (FIG. 1, panel A). Mice were implanted subcutaneously and treatment began on Day 5. Treatments ended on Day 25 after three weeks of treatment. Treatment with Compound 1 (15 mg/kg, PO, BID) produced a statistically significant tumor growth delay of 17.8 days (p<0.05), and a Day 11% T/C of 16% (p<0.05). Treatment with Compound 1 (7.5 mg/kg, PO, BID) produced a statistically significant tumor growth delay of 11.4 days (p<0.05), and a Day 11% T/C of 22% (p<0.05). Treatment with Compound 1 (3.75 mg/kg, PO, BID) produced a tumor growth delay of 4.5 days and a Day 11% T/C of 39%, which was not statistically significant at this dose.

Compound 1 was evaluated for inhibition of NTRK1 phosphorylative activation after a single dose in the KM-12 TPM3-NTRK1 xenograft model (FIG. 1, panel B). Mice were implanted subcutaneously and a single treatment was given on Day 8. At time points after the single oral dose, tumors were resected, frozen, pulverized, and then lysed. Lysates were separated by SDS-PAGE and protein was transferred to PVDF. Phospho-NTRK1 (Tyr674/675) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Blots were stripped and probed for total NTRK1 using an antibody from Santa Cruz Biotech (Santa Cruz, Calif.). Oral administration of a single dose of Compound 1 (15 mg/kg; PO) afforded ˜95% inhibition of NTRK1 kinase phosphorylation in vivo through 18 hours post dose. Compound 1 exhibited 79% inhibition of NTRK1 phosphorylation at 24 hours post dose. At a single oral dose of 7.5 mg/kg, Compound 1 inhibited NTRK1 phosphorylation by >95% for 8 hours. At 12, 18, and 24 hours post dose, Compound 1 exhibited 66%, 74%, and 59% inhibition of NTKR1 phosphorylation, respectively.

### Example 10

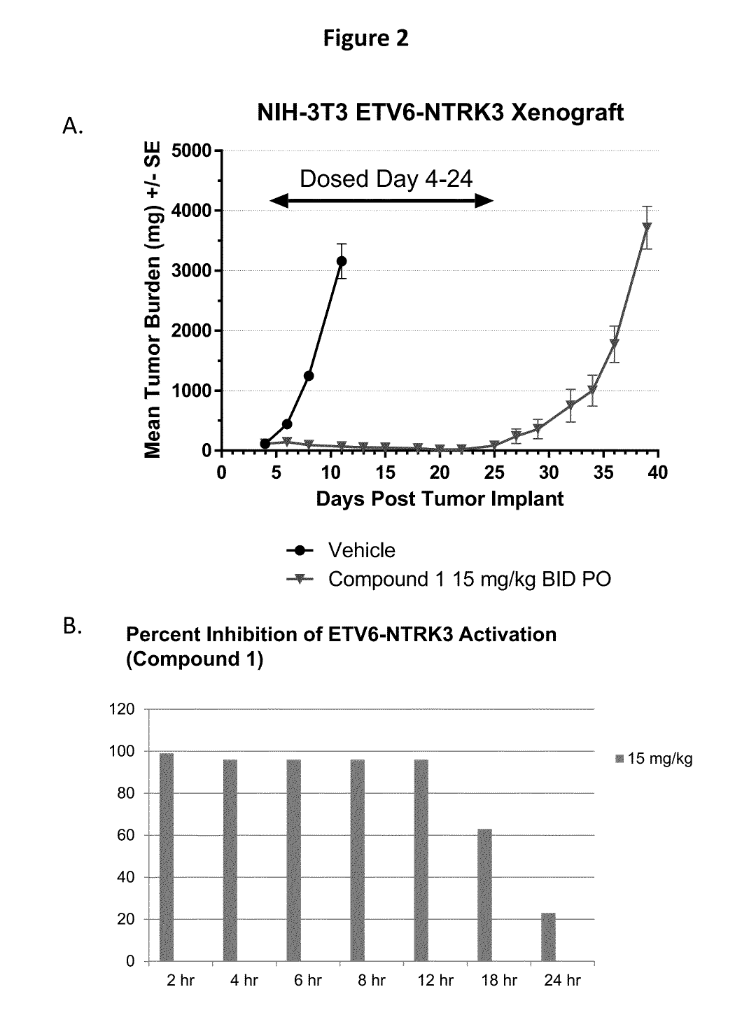

### Evaluation of Compound 1 in the ETV6-NTRK3 Transformed NIH-3T3 Xenograft Model (FIG. 2)

Compound 1 was evaluated for single-agent efficacy in the transfected NIH-3T3 ETV6-NTRK3 xenograft model (FIG. 2, panel A). Mice were implanted subcutaneously and treatment began on Day 4. Treatments ended on Day 24 after three weeks of treatment. Treatment with Compound 1 (15 mg/kg; PO; BID) produced a statistically significant tumor growth delay of 26.5 days (p<0.05) and Day 8% T/C of 6% (p<0.05).

Compound 1 was evaluated for inhibition of NTRK3 phosphorylative activation after a single dose in the transfected NIH-3T3 ETV6-NTRK3 xenograft model (FIG. 2, panel B). Mice were implanted subcutaneously and a single treatment was given on Day 6. At time points after the single oral doses, tumors were resected, frozen, pulverized, and then lysed. Lysates were separated by SDS-PAGE and protein was transferred to PVDF. Phospho-NTRK3 (Tyr516) was detected using an antibody from Cell Signaling Technology (Beverly, Mass.), ECL Plus detection reagent (GE Healthcare, Piscataway, N.J.) and a Molecular Devices Storm 840 phosphorimager in fluorescence mode. Administration of a single dose of Compound 1 (15 mg/kg; PO) afforded >95% inhibition of NTRK3 kinase phosphorylation in vivo through 12 hours post dose. Compound 1 exhibited 63% inhibition of NTRK3 phosphorylation at 18 hours and 23% inhibition of NTRK3 phosphorylation at 24 hours post dose.

Those skilled in the art will recognize, or be able to ascertain, using no more than routine experimentation, numerous equivalents to the specific embodiments described specifically in this disclosure. Such equivalents are intended to be encompassed in the scope of the following claims.

### REFERENCES

Alberti, L., Carniti, C., Miranda, C., Roccato, E. & Pierotti, M. A. J. Cell Physiol. 195, 168-186 (2003).
Brodeur G M, Minturn J E, Ho R, Simpson A M, et al. Trk Receptor Expression and Inhibition in Neuroblastomas. Clin Cancer Res 2009; 15: 3244.
Cuesta, L F. Abstract 1531: Cross-entity mutation analysis of lung neuroendocrine tumors sheds light into their molecular origin and identifies new therapeutic targets. DOI: 10.1158/1538-7445.AM2014-1531 Conference: American Association for Cancer Research Annual Meeting 2014, At San Diego, USA, Volume: 1531
Greco, A., Miranda, C. & Pierotti, M. A. Rearrangements of NTRK1 gene in papillary thyroid carcinoma. Mol. Cell. Endocrinol. 321, 44-49 (2010).
Kim J, Lee Y, Cho H J, Lee Y E, An J, Cho G H, Ko Y H, Joo K M, Nam D H. NTRK1 fusion in glioblastoma multiforme. PLoS One. 2014 Mar. 19; 9(3):e91940.
Knezevich, S. R., McFadden, D. E., Tao, W., Lim, J. F. & Sorensen, P. H. A novel ETV6-NTRK3 gene fusion in congenital fibrosarcoma. Nat. Genet. 18, 184-187 (1998).
Liu D, Zhang Y, Dang C, Ma Q, LEE W, et al. siRNA directed against TrkA sensitizes human pancreatic cancer cells to apoptosis induced by gemcitabine through an inactivation of PI3K/Akt-dependent pathway. Oncology Reports 2007; 18:673-677.
Martin-Zanca, D., Hughes, S. H. & Barbacid, M. Nature 1986; 319: 743-748.
Patel M. Clinical responses to entrectinib in a patient with NTRK1-rearranged non small cell lung cancer. Intl Association for the study of lung cancer. Sep. 7, 2015.
Ricarte-Filho J C, Li S, Garcia-Rendueles M E R, et al. Identification of kinase fusion oncogenes in post-Chernobyl radiation-induced thyroid cancers. J Clin Invest. 2013; 123(11):4935-4944.
Ross, J. S. et al. New routes to targeted therapy of intrahepatic cholangiocarcinomas revealed by next-generation sequencing. Oncologist 2014; 19: 235-242.
Reuther G W, Lambert Q T, Caligiuri M A, Der C J. Identification and Characterization of an Activating TrkA Deletion Mutation in Acute Myeloid Leukemia. Molecular and Cellular Biology. 2000; 20:8655-8666.
Schindler, T, Bornmann, W, Pellicena, P, Miller, W T, Clarkson, B, Kuriyan, J. Structural mechanism for STI-571 inhibition of Abelson tyrosine kinase. Science 2000; 289: 1938-42.
Stransky N, Cerami E, Schalm S, Kim J L, et al. The landscape of kinase fusions in cancer. Nature Communications 2014; Sep. 10:1-10.
Tognon, C. et al. Expression of the ETV6-NTRK3 gene fusion as a primary event in human secretory breast carcinoma. Cancer Cell 2002; 2: 367-376.
Vaishnavi A, Le A T, and Doebele R C. TRKing down an old oncogene in a new era of targeted therapy. Cancer Discovery 2015; January; 5(1):25-34.
Vaishnavi A, Capelletti M, Le A T, Kako S, et al. Oncogenic and drug-sensitive NTRK1 rearrangements in lung cancer. Nature Medicine 2013; 19:1469-1472.
Wiesner T, He J, Yelensky R, Esteve-Puig R, et al. Kinase fusions are frequent in Spitz tumours and spitzoid melanomas. Nat Commun. 2014; 5: 3116.
Zhu Z, Friess H, diMola F F, Zimmermann A, et al. Nerve Growth Factor Expression Correlates With Perineural Invasion and Pain in Human Pancreatic Cancer. Journal of Clinical Oncology, 1999; 17:2419-2428.

1. A method of inhibiting TRK kinase mediated tumor growth, survival, or disease progression comprising administering to a subject in need thereof an effective amount of N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof.

2. The method of claim 1, wherein tumor growth, survival, or progression is caused by an overexpression of a TRK kinase, mutation of a TRK kinase, or a TRK kinase fusion protein.

3. The method of claim 1, wherein tumor growth, survival, or progression is caused by a NTRK1 fusion protein.

4. The method of claim 3, wherein the NTRK1 fusion protein is selected from MPRIP-NTRK1, CD74-NTRK1, RFWD2-NTRK1, SQSTM1-NTRK1, TPM3-NTRK1, TFG-NTRK1, TPR-NTRK1, RABGAP1L-NTRK1 LMNA-NTRK1, TP53-NTRK1, NFASC-NTRK1, or PEAR1-NTKR1.

5. The method of claim 1, wherein tumor growth, survival, or progression is caused by a NTRK2 fusion protein.

6. The method of claim 5, wherein the NTRK2 fusion protein is selected from PAN3-NTRK2, AFAP1-NTRK2, TRIM24-NTRK2, QK1-NTRK2, NACC2-NTRK2, VCL-NTRK2, or AGBL4-NTRK2.

7. The method of claim 1, wherein tumor growth, survival, or progression is caused by a NTRK3 fusion protein.

8. The method of claim 7, wherein the NTRK3 fusion protein is selected from ETV6-NTRK3 or BTBD1-NTRK3.

9. The method of claim 1, wherein tumor growth, survival, or progression is caused by a mutation in a TRK kinase.

10. The method of claim 9, wherein the mutation in a TRK kinase is an NTRK1 deletion mutation in acute myeloid leukemia.

11. The method of claim 1, wherein tumor growth, survival, or progression is caused by overexpression of a wild-type TRK kinase.

12. The method of claim 11, wherein NTRK1 is overexpressed in pancreatic cancer or neuroblastoma.

13. The method of claim 1, wherein the tumor is lung adenocarcinoma, cholangiocarcinoma, colorectal cancer, colon adenocarcinoma, papillary thyroid cancer, spitzoid neoplasms, glioblastoma, sarcomas, congenital fibrosarcoma, astrocytomas, head and neck cancer, low grade glioma, secretory breast cancer, acute myeloid leukemia, congenital mesoblastic nephroma, acute lymphoblastic leukemia, thyroid carcinoma, cutaneous melanoma, pediatric glioma, neuroblastoma, or pancreatic cancer.

14. The method of claim 1, wherein N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof is administered as a single agent or in combination with other cancer targeted therapeutic agents, cancer-targeted biologicals, or chemotherapeutic agents.

15. A method of treating cancer in a subject in need thereof comprising comprising administering to the subject an effective amount of N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof, wherein the cancer is associated with overexpression of a TRK kinase, mutation of a TRK kinase, and/or a TRK kinase fusion protein.

16. The method of claim 15, wherein the cancer is caused by a NTRK1 fusion protein.

17. The method of claim 16, wherein the NTRK1 fusion protein is selected from MPRIP-NTRK1, CD74-NTRK1, RFWD2-NTRK1, SQSTM1-NTRK1, TPM3-NTRK1, TFG-NTRK1, TPR-NTRK1, RABGAP1L-NTRK1 LMNA-NTRK1, TP53-NTRK1, NFASC-NTRK1, or PEAR1-NTKR1.

18. The method of claim 15, wherein the cancer is caused by a NTRK2 fusion protein.

19. The method of claim 18, wherein the NTRK2 fusion protein is selected from PAN3-NTRK2, AFAP1-NTRK2, TRIM24-NTRK2, QK1-NTRK2, NACC2-NTRK2, VCL-NTRK2, or AGBL4-NTRK2.

20. The method of claim 15, wherein the cancer is caused by a NTRK3 fusion protein.

21. The method of claim 20, wherein the NTRK3 fusion protein is selected from ETV6-NTRK3 or BTBD1-NTRK3.

22. The method of claim 15, wherein the cancer is caused by a mutation in a TRK kinase.

23. The method of claim 22, wherein the mutation is a NTRK1 deletion mutation in acute myeloid leukemia.

24. The method of claim 15, wherein the cancer is caused by overexpression of a wild-type TRK kinase.

25. The method of claim 24, wherein NTRK1 is overexpressed in pancreatic cancer or neuroblastoma.

26. The method of claim 15, wherein the cancer is lung adenocarcinoma, cholangiocarcinoma, colorectal cancer, colon adenocarcinoma, papillary thyroid cancer, spitzoid neoplasms, glioblastoma, sarcomas, congenital fibrosarcoma, astrocytomas, head and neck cancer, low grade glioma, secretory breast cancer, acute myeloid leukemia, congenital mesoblastic nephroma, acute lymphoblastic leukemia, thyroid carcinoma, cutaneous melanoma, pediatric glioma, neuroblastoma, or pancreatic cancer.

27. The method of claim 15, wherein N-(4-(2-(cyclopropanecarboxamido)pyridin-4-yloxy)-2,5-difluorophenyl)-N′-(4)cyclopropane-1,1-dicarboxamide, or a pharmaceutically acceptable salt thereof is administered as a single agent or in combination with other cancer targeted therapeutic agents, cancer-targeted biologicals, or chemotherapeutic agents.


SEQUENCE LISTING (3 sequences)

SEQ ID NO: 1 - protein, 381 aa, Homo sapiens
MKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLGKGVEACQLGTDDYDIPTTHHHHHH

SEQ ID NO: 2 - protein, 357 aa, Homo sapiens
MVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILGKGGRADPAFLYKVVRMNEDLGKPIPNPLLGLDSTRTGHHHHHH

SEQ ID NO: 3 - protein, 360 aa, Homo sapiens
MVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILGKGGRADPAFLYKVVRMNEDLGKPIPNPLLGLDSTRTGHHHHHH